(19) United States
(12) Patent Application Publication
(10) Pub. No.: US 2024/0285364 A1
(43) Pub. Date: Aug. 29, 2024

(54) MEDICAL MEASUREMENT APPARATUS

(71) Applicant: Konica Minolta Inc., Tokyo (JP)

(72) Inventors: Tatsushi CHIHARA, Tokyo (JP); Takahiko SHIRAISHI, Tokyo (JP)

(21) Appl. No.: 18/582,766

(22) Filed: Feb. 21, 2024

(30) Foreign Application Priority Data
Feb. 28, 2023 (JP) 2023-029565

(51) Int. Cl. A61B 50/20; A61B 8/00; A61M 25/02

(52) U.S. Cl. CPC A61B 50/20; A61B 8/4405; A61B 8/4427; A61B 2562/222; A61M 2025/028

(57) ABSTRACT

A medical measurement apparatus includes: a cable connected to a probe; an apparatus body from which the cable is led out, the apparatus body having a bottom face and a side face larger than the bottom face, and being vertically long in shape in use; and a hook disposed on the apparatus body, the hook being lower than a position of a center of gravity of the apparatus body, the hook holding the cable by hooking.

### CROSS REFERENCE TO RELATED APPLICATIONS

The present invention claims priority under 35 U.S.C. § 119 to Japanese Application, 2023-029565, filed on Feb. 28, 2023, the entire contents of which being incorporated herein by reference.

### BACKGROUND

### Technological Field

The present invention relates to a medical measurement apparatus including a cable connected to a probe.

### Description of the Related Art

As one of medical measurement apparatuses, known has been a diagnostic ultrasound apparatus that transmits an ultrasound wave toward a subject, receives a reflected wave thereof, and performs predetermined signal processing on a reception signal to visualize the shape, property, or dynamics inside the subject as an ultrasound image. Such a diagnostic ultrasound apparatus can acquire an ultrasound image resulting from a simple operation of applying an ultrasound probe connected to the apparatus body through a cable to the body surface of the subject or inserting the ultrasound probe into the body thereof. Thus, the diagnostic ultrasound apparatus is safe and the burden on the subject is small.

As a diagnostic ultrasound apparatus, for example, as disclosed in JP 2005-526565 A, an ultrasound system provided integrally with a cart has been known. In this system, a probe is connected through a cable led out upward from an ultrasound apparatus body provided integrally with the cart. Operation of the cart causes the ultrasound system itself to move easily that enables ultrasound diagnosis.

In the system of JP 2005-526565 A, the cable is hooked to the handle of the cart through an opening with which the center of the handle of the cart is provided, so that the probe can be used while preventing the cable from getting entangled or the cable from being caught in the caster of the cart.

Meanwhile, as a diagnostic ultrasound apparatus, known has been a diagnostic ultrasound apparatus including a diagnostic ultrasound apparatus body provided in a cart and separable from the cart. The diagnostic ultrasound apparatus that is made compact and slim and vertically long in shape can be used independently and upright. With this apparatus, for example, even for a subject at a place where the cart cannot be moved, the user can carry and move the diagnostic ultrasound apparatus body to perform diagnosis, at the movement destination, with a probe connected to the diagnostic ultrasound apparatus body.

However, in use of the diagnostic ultrasound apparatus independent and upright, as a disadvantage, the slim diagnostic ultrasound apparatus body disposed upright is pulled through the cable due to movement of the probe, so that the ultrasound diagnostic apparatus body falls depending on the force and the work cannot be performed safely.

### SUMMARY

The present invention has been made in view of the above points, and an object of the present invention is to provide a medical measurement apparatus that enables safety work without falling even if a probe is used.

To achieve the abovementioned object, according to an aspect of the present invention, a medical measurement apparatus reflecting one aspect of the present invention comprises: a cable connected to a probe; an apparatus body from which the cable is led out, the apparatus body having a bottom face and a side face larger than the bottom face, and being vertically long in shape in use; and a hook disposed on the apparatus body, the hook being lower than a position of a center of gravity of the apparatus body, the hook holding the cable by hooking.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a perspective view of a measurement system including a diagnostic ultrasound apparatus as an exemplary measurement apparatus according to an embodiment of the present invention;

FIG. 2 is an enlarged perspective view of the diagnostic ultrasound apparatus as the exemplary measurement apparatus according to the embodiment of the present invention;

FIG. 3 is an enlarged perspective view of a hook of the diagnostic ultrasound apparatus as the exemplary measurement apparatus according to the embodiment of the present invention;

FIG. 4 is a view illustrating the hook being attached of the diagnostic ultrasound apparatus as the exemplary measurement apparatus according to the embodiment of the present invention;

FIG. 5 is a front view of the hook of the diagnostic ultrasound apparatus as the exemplary measurement apparatus according to the embodiment of the present invention;

FIG. 6 is a schematic view illustrating a position of the hook on the diagnostic ultrasound apparatus;

FIGS. 7A and 7B are schematic views each illustrating the position of the hook on the diagnostic ultrasound apparatus;

FIG. 8 is a perspective view illustrating Modification 1 to the hook; and

FIG. 9 is a perspective view illustrating Modification 2 to the hook.

### DETAILED DESCRIPTION OF EMBODIMENTS

Hereinafter, one or more embodiments of the present invention will be described with reference to the drawings. However, the scope of the invention is not limited to the disclosed embodiments.

### <Diagnostic Ultrasound System (Measurement System) 1>

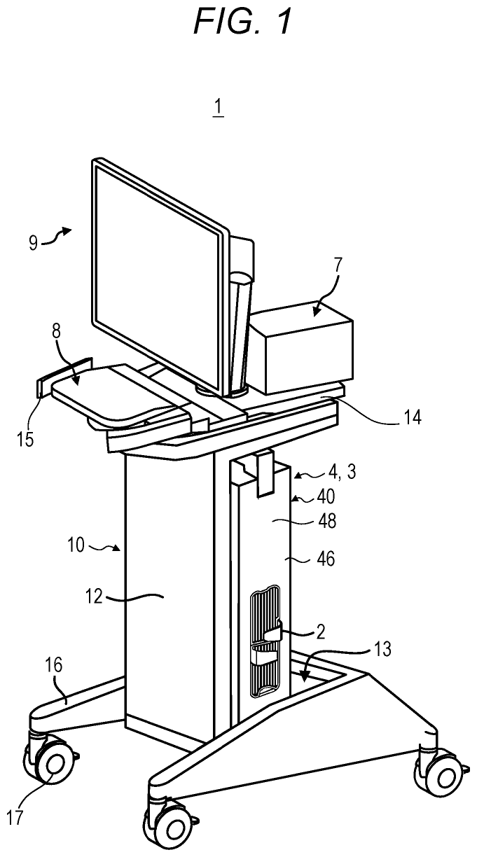

FIG. 1 is a perspective view of a measurement system including a measurement apparatus according to an embodiment of the present invention.

A diagnostic ultrasound apparatus 3 illustrated in FIG. 1 is an exemplary measurement apparatus. The diagnostic ultrasound apparatus 3 is mounted on a cart 10 and included, together with the cart 10, in a diagnostic ultrasound system 1.

The diagnostic ultrasound system 1 includes the diagnostic ultrasound apparatus 3, a personal computer (PC) 7, a monitor 9, and the cart 10.

### <Cart 10>

The cart 10 includes a placement portion 13 on which the diagnostic ultrasound apparatus body (hereinafter, referred to as an “apparatus body”) 4 is placed. The placement portion 13 is provided on a lower moving portion provided with casters 17. The lower moving portion includes legs 16 protruding in four directions around the placement portion 13. Each leg 16 has a distal end with the corresponding caster 17 attached thereto so as to be movable in all directions by 360°.

The cart 10 includes an apron 12, a top face portion 14, and a handle 15. The apron 12 extends upward from the lower moving portion and has a rear face where the apparatus body 4 placed on the placement portion 13 is located.

The apron 12 has an upper portion with the top face portion 14 attached thereon so as to extend horizontally. Together with the placement portion 13 and the top face portion 14, the apron 12 is included in an accommodating portion that accommodates the apparatus body 4 upright.

The PC 7 serving as an analysis device is disposed on the top face portion 14, and a frame-shaped portion for operating the cart 10 from the rear is disposed around the top face portion 14.

Further, the top face portion 14 has a part extending to the front-end side (front face side) of the top face portion 14. A keyboard 8 is disposed on the part. The cart 10 is provided with the handle 15 protruding to the front-end side of the top face portion 14, and the cart 10 is easily movable from the front and rear sides together with the frame-shaped portion on the rear-end side.

### <Diagnostic Ultrasound Apparatus 3>

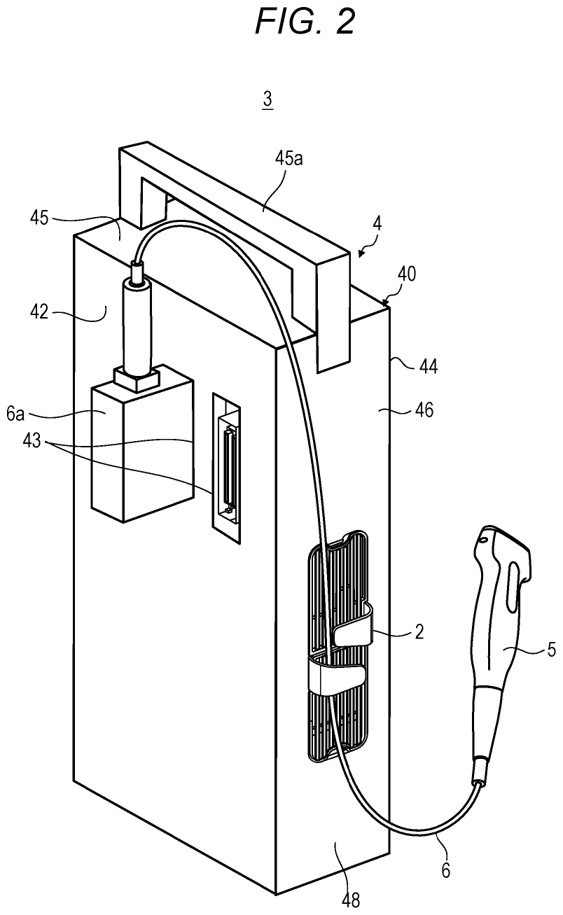

FIG. 2 is an enlarged perspective view of the diagnostic ultrasound apparatus as an exemplary measurement apparatus according to the embodiment of the present invention. Note that, in the present embodiment, expressions related to directions such as up, down, left, right, front, rear, front face side, and rear face side used to describe the configuration and operation of each component of the ultrasound system and the diagnostic ultrasound apparatus are not absolute but relative. These expressions are appropriate in a case where each component of the ultrasound system and the diagnostic ultrasound apparatus has the posture illustrated in the drawings. However, in a case where the posture changes, these expressions should be interpreted as being changed in accordance with the change in posture.

The diagnostic ultrasound apparatus 3 is used to visualize the shape, property, or dynamics in the subject as an ultrasound image and perform image diagnosis. For example, the diagnostic ultrasound apparatus 3 may display a brightness mode (B-mode) image on the monitor (display) 9 connected thereto or may display a color flow mapping (CFM) image obtained by a color Doppler method on the B-mode image in a superimposed manner.

The diagnostic ultrasound apparatus 3 includes the apparatus body 4, an ultrasound probe (hereinafter, also referred to as “probe”) 5, and a cable 6.

The apparatus body 4 and the probe 5 are electrically connected to each other by fitting the connector 6a of the cable 6 in the socket 43 of the apparatus body 4.

The probe 5 transmits an ultrasound wave to a subject as a diagnosis target and receives an ultrasound echo reflected on the subject. The probe 5 convers the ultrasound echo into a reception signal to transmit the reception signal to the apparatus body 4.

The probe 5 is compatible with electronic scanning, and for example, a linear probe, a convex probe, or a sector probe is applicable. In the present embodiment, the probe 5 is a convex probe capable of corresponding to a wider diagnostic region.

The apparatus body 4 includes a hook 2, a housing 40, and a vent hole 48. The hook 2 and the vent hole 48 are disposed on the housing 40.

The apparatus body 4 is made compact and slim. The housing 40 is a vertically-long rectangular parallelepiped having a rectangular parallelepiped shape of which height is longer than the width and thickness. The apparatus body 4 is independently disposed upright. Specifically, the apparatus body 4 is placed upright in the placement portion 13 of the cart 10 in an upright posture illustrated in FIG. 2 or is disposed outside the cart 10 for use.

The connector 6a of the cable 6 is fitted in the socket 43 provided on the front face portion (frontal face portion) 42 of the housing 40, and the cable 6 is attached to the apparatus body 4 so as to be led out upward the apparatus body 4.

Typically, the cable 6 led out from the diagnostic ultrasound apparatus body is disposed so as to be led out upward for a user to use and operate easily.

The housing 40 has an upper face 45 on which a handle 45a protruding upward is disposed and has a left-side face 46 on which the hook 2 and the vent hole 48 are disposed.

Gripping of the handle 45a enables the apparatus body 4 to be lifted easily, leading to easy carry of the apparatus body 4.

### <Hook 2>

In the present embodiment, the hook 2 is a cable hook for hooking the cable 6 as the probe cable led out from the apparatus body 4. In response to pulling of the cable 6, the hook 2 receives a load applied to the connector 6a as a proximal end of the cable 6 or the socket 43 in which the connector 6a is fitted.

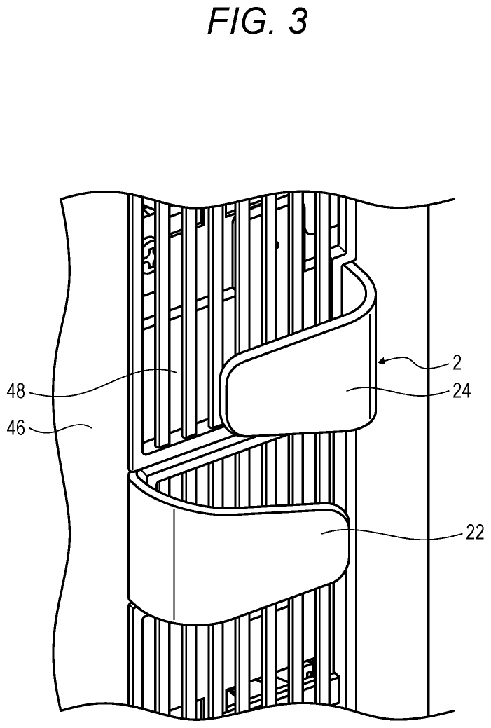

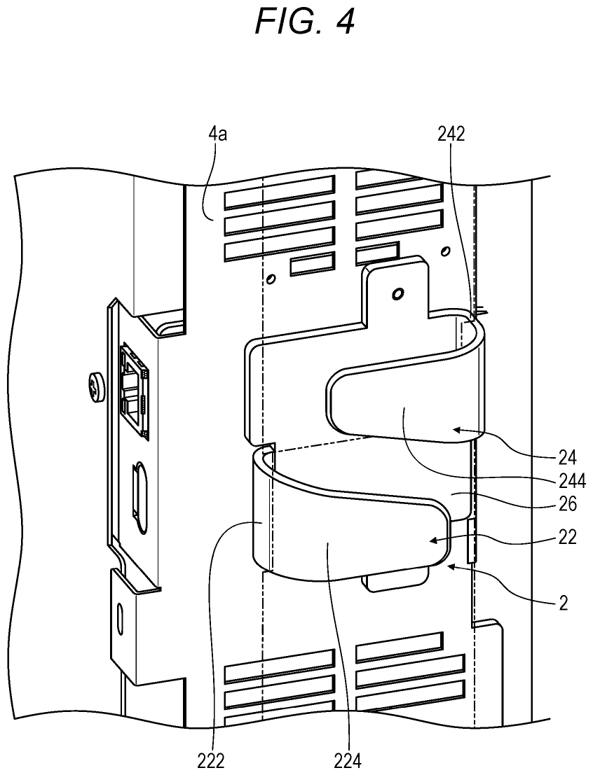

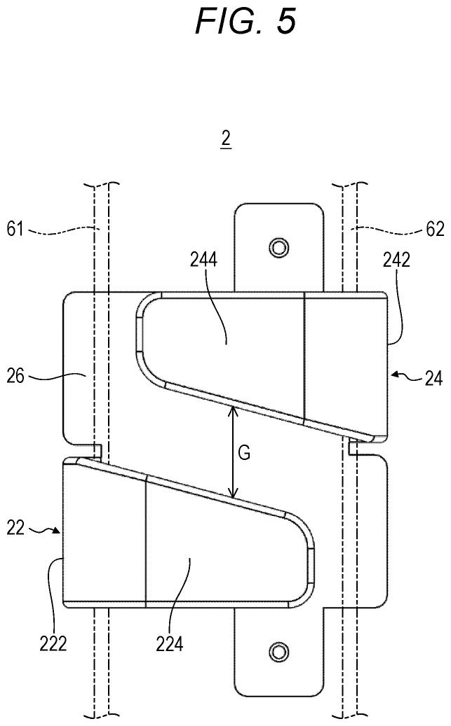

FIG. 3 is an enlarged perspective view of the hook of the diagnostic ultrasound apparatus as the exemplary measurement apparatus according to the embodiment of the present invention. FIG. 4 is a view illustrating the hook being attached. FIG. 5 is a front view of the hook.

The hook 2 includes an arm 22 and an arm 24 each protruding from the left-side face 46.

The left-side face 46 has, in the front and rear direction, a side portion and another side portion spaced apart from each other. The arm 22 and the arm 24 protrude, respectively, from a position of the side portion and a position of the other side portion and are bent so as to be close to each other, in which the positions are different in the vertical direction. Thus, the arm 22 and the arm 24 each have its distal end serving as a free end.

The arms 22 and 24 and are spaced apart from each other with a gap G in the vertical direction and extend from the front and rear sides of the left-side face 46 so as to laterally cover the left-side face 46.

As illustrated in FIGS. 4 and 5, the hook 2 includes the arms 22 and 24 and a fastening plate 26 from which the arms 22 and 24 protrude, as an example.

The hook 2 is fastened, through a fastening plate 26, by a fixing member such as a screw to a body side face 4a of the apparatus body 4 with a cover of the vent hole 48 removed therefrom.

The fastening plate 26 has a space of which the width is a predetermined width as a length in the front and rear direction, the width enabling the cable 6 to pass through, and the arms 22 and 24 are bent from both ends of the fastening plate 26.

The arms 22 and 24 include protruding extension portions 222 and 242 and holding extension portions 224 and 244, respectively. The holding extension portions 224 and 244 are bent and extend from the distal ends of the protruding extension portions 222 and 242 along the surface of the fastening plate 26. Thus, the holding extension portions 224 and 244 each have its distal end serving as a free end.

The arms 22 and 24 are formed in an L shape by the protruding extension portions 222 and 242 and the holding extension portions 224 and 244.

The protruding extension portions 222 and 242 are bent from the positions different in the vertical direction from the both ends of the fastening plate 26, and vertically extend from the fastening plate 26.

When the cable 6 is hooked to the hook 2 formed in this manner, the cable 6 is located within a region surrounded by the protruding extension portions 222 and 242 and the holding extension portions 224 and 244 through the gap G between the arms 22 and 24.

The gap G is larger than the diameter of the cable 6, and the cable 6 can be inserted. Note that the arms 22 and 24 forming the gap G may be deformed such that the cable 6 can be inserted inside.

Further, the arms 22 and 24 are disposed such that the gap G is extends in a slant direction intersecting the extending direction of the cable 6.

With this arrangement, for example, even if the cable 6 moves in the vertical direction from the left-side face 46 to come off, the cable 6 is hooked to the holding extension portions 224 and 244 and hardly comes off. Further, even in movement in the front and rear direction on the left-side face 46, namely, even in movement in the front and rear direction (the left and right direction on the drawing plane in FIG. 5) to the position indicated by a cable 61 as illustrated in FIG. 5, the cable 61 is hooked to the protruding extension portion 222, or even in movement in the front and rear direction (the left and right direction on the drawing plane in FIG. 5) to the position indicated by a cable 62 as illustrated in FIG. 5, the cable 62 is hooked to the protruding extension portion 242. Thus, the cable 61 and the cable 62 hardly come off.

Further, because the gap G extends at a slant, the cable 6 is easily inserted and located inside the arms 22 and 24, namely, inside the hook 2 through the gap G, and hardly comes off the inside.

With this arrangement, the cable 6 that is pulled and moved is hooked to the hook 2 and the hook 2 receives the load at that time, resulting in reduction of the load applied to the connector 6a as the proximal end of the cable 6 or the socket 43 in which the connector 6a is fitted.

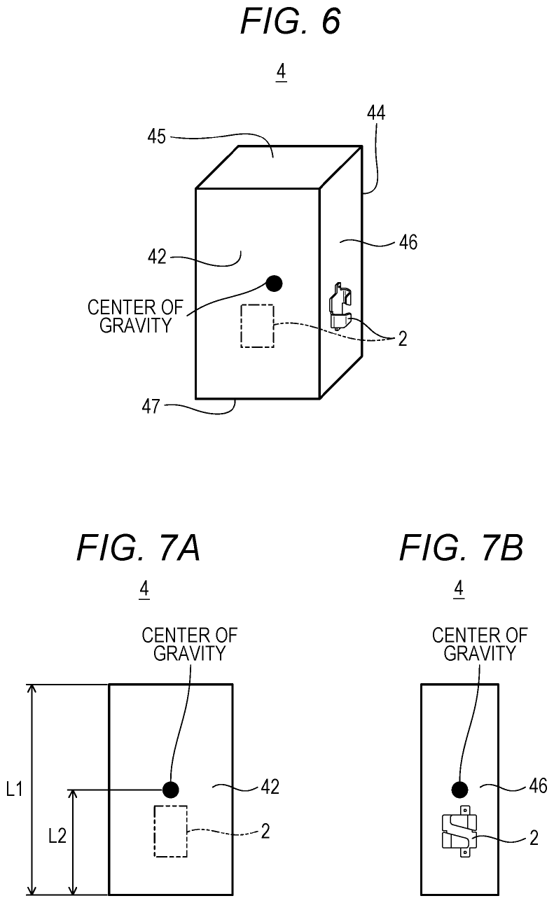

FIGS. 6, 7A, and 7B are schematic views illustrating the position of the hook of the diagnostic ultrasound apparatus.

The apparatus body 4, specifically, the housing 40 has an outer shape of an elongated rectangular parallelepiped having an area of bottom face 47<side faces (front face, rear face, left-side face, and right-side face) 42, 44, and 46. That is, the side faces are each longer in the longest length than the bottom face 47, and the apparatus body 4 is used in a vertically long and upright posture as the entirety of the housing 40.

In such an apparatus body 4, as illustrated in FIG. 6, the hook 2 that holds the cable 6 is disposed below the position of the center of gravity of the apparatus body 4.

Therefore, in order to operate the probe 5 by the user, even if the cable 6 is pulled out beyond the extra length of the cable 6, the cable 6 acts to pull the apparatus body 4 while being caught by the hook 2, and diagnosis can be performed safely without falling of the apparatus body 4.

Note that as indicated by a two-dot chain line in FIG. 6, the hook 2 may be provided at any part lower than the position of the center of gravity of apparatus body 4.

In order to hook to the hook 2, the cable 6 is hooked below the center of gravity of the apparatus body 4 when the cable 6 extending from above to below inside the hook 2 moves upward. As a result, even if the distal end of the cable 6 moves upward, the apparatus body 4 is not pulled at an upper portion, and the apparatus body 4 can be prevented from falling.

As illustrated in FIGS. 7A and 7B, the position of the center of gravity P of the apparatus body 4 is located on a substantially central portion of the length of height L1 of the apparatus body 4. The substantially central portion is within 10% above and below the position of the central position of height L1 and satisfies P=L2±(0.1×L), where L2=L/2. The position of the center of gravity is within the range, in the vertical direction, of which the center is identical to the center of the height of the apparatus body 4 and the range has an upper half and a lower half each identical to 10% of the height of the apparatus body 4. The hook 2 is disposed within the range. The hook 2 disposed at the position can more suitably prevent falling of the apparatus body 4.

According to the diagnostic ultrasound apparatus 3 of the present embodiment, the hook 2 is disposed below the position of the center of gravity of the apparatus body 4, and the cable 6 is not detached from the hook 2 even if the cable 6 is moved through the hook 2.

As a result, in operation of the probe 5, the user can work safely without falling of the apparatus body 4 even if the cable 6 is pulled out beyond the extra length thereof.

Note that the hook 2 may have any shape as long as the cable 6 being hooked is hardly detached and the hook 2 is disposed at a part (lower portion) below the center of gravity of the apparatus body 4. Such an example is described in Modification 1 and Modification 2.

### Modification 1

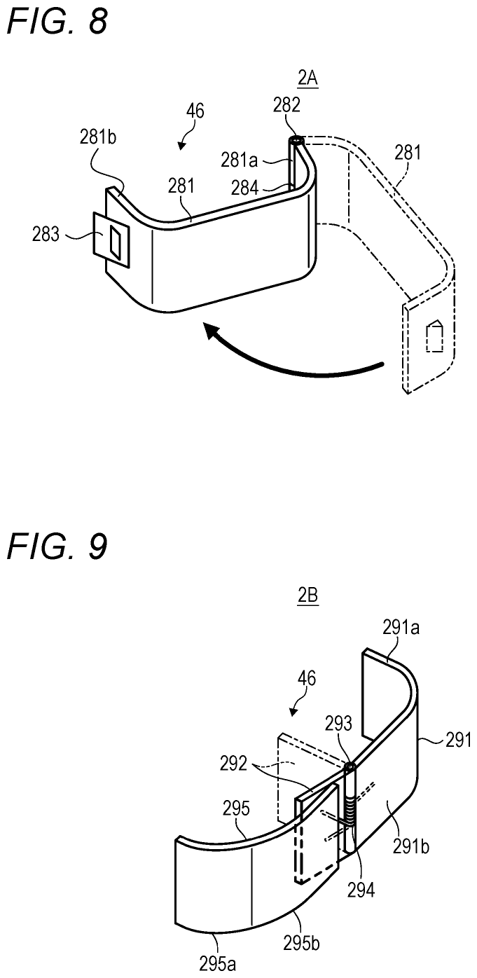

FIG. 8 is a perspective view illustrating Modification 1 to the hook.

For example, a hook 2A illustrated in FIG. 8 is provided on the left-side face 46 (see FIG. 2) of the apparatus body 4 so as to be opened and closed. The hook 2A includes a U-shaped hook body 281 that forms a rectangular space together the left-side face 46, and a lock receiver (engagement part) 283 that the hook body 281 is engaged with. The hook body 281 has an end 281a at which the hook body 281 is turnably attached to a hinge receiver 284 of the left-side face 46 through a hinge 282. The hook body 281 has another end 281b having an engagement claw 285 to be engaged with the lock receiver 283.

The hook body 281 and the lock receiver 283 protrude from the left-side face 46 of the apparatus body 4. Due to detachable engagement of the engagement claw 285 with the lock receiver 283, the hook body 281 openably and closably form, together with the left-side face 46, a region through which the cable 6 passes. Thus, the cable 6 is located within the region, and can be hooked to the hook body 281.

### Modification 2

FIG. 9 is a perspective view illustrating Modification 2 to the hook.

A hook 2B illustrated in FIG. 9 includes a U-shaped hook body having an openable and closable part. The hook 2B includes the U-shaped hook body that forms an insertion region through which the cable 6 is inserted, together with the left-side face 46 as a side face of the apparatus body 4. The hook body includes L-shaped arms 291 and 295 and has a part (between the arms 291 and 295) at which a flap 292 is provided openably and closably. The flap 292 moves from the side of location of the insertion region to enclose the insertion region and moves from outside to release the insertion region.

Specifically, the L-shaped arms 291 and 295 are disposed in a U shape with a gap. The arm 291 includes a protruding portion 291a having an end and an extending portion 291b. The arm 295 includes a protruding portion 295a having an end and an extending portion 295b. The protruding portions 291a and 295a face to each other with a predetermined interval from the left-side face 46 (see FIG. 2) of the apparatus body 4. The extending portions 291b and 295b are bent and extend from the respective ends of the protruding portions 291a and 295a so as to close to each other.

The extending portion 291b of the arm 291 has a distal end provided with a flap 292 operable and closable a gap between the extending portion 291b of the arm 291 and the extending portion 295b of the arm 295. The flap 292 is biased by a biasing member 294 so as to abut on the extending portion 295b, namely, so as to close the gap.

In order to hook the cable 6 to the hook 2B, the cable 6 is inserted through the gap formed due to movement of the flap 292 against the biasing force. As a result, the cable 6 is less likely to come off the hook 2 and can be easily hooked to the hook 2.

Note that, although the measurement apparatus is a diagnostic ultrasound apparatus, the present invention is not limited thereto, and any measurement apparatus may be used as long as it is a measurement apparatus to which a cable provided with a probe for performing, for example, diagnosis, inspection, and measurement is connected. For example, the measurement apparatus is an apparatus different from the diagnostic ultrasound apparatus and is also applicable to a medical instrument such as an electrocardiograph.

The measurement apparatus according to the present invention has an effect of being able to work safely without falling even if a probe is used and is mainly useful for a medical instrument including a cable to which a probe is connected.

The embodiments of the present invention have been described above. Although embodiments of the present invention have been described and illustrated in detail, the disclosed embodiments are made for purposes of illustration and example only and not limitation. That is, the description of the configuration of the apparatus and the shape of each component are examples, and thus it is obvious that various modifications and additions can be made to these examples within the scope of the present invention. The scope of the present invention should be interpreted by terms of the appended claims.

What is claimed is:

1. A medical measurement apparatus comprising:
a cable connected to a probe;
an apparatus body from which the cable is led out, the apparatus body having a bottom face and a side face larger than the bottom face, and being vertically long in shape in use; and
a hook disposed on the apparatus body, the hook being lower than a position of a center of gravity of the apparatus body, the hook holding the cable by hooking.

2. The medical measurement apparatus according to claim 1,
wherein the position of the center of gravity is within a range, in a vertical direction, of which a center is identical to a center of a height of the apparatus body, the range having an upper half and a lower half each identical to 10% of the height of the apparatus body.

3. The medical measurement apparatus according to claim 1, wherein
the hook includes a pair of arms being each L-shaped,
the pair of arms being spaced apart from each other in a direction orthogonal to a vertical direction on a side face of the apparatus body, the pair of arms protruding from positions different in the vertical direction and extending closer to each other from position of the protruding, and
the pair of arms are disposed with a gap, in the vertical direction, larger than a diameter of the cable.

4. The medical measurement apparatus according to claim 3,
wherein the pair of arms are disposed such that the gap extends at a slant with respect to the vertical direction.

5. The medical measurement apparatus according to claim 1, wherein
the hook is disposed on any of four side faces of the apparatus body.

6. The medical measurement apparatus according to claim 1, wherein
the hook includes:
a hook body being U-shaped, an end of the hook body being turnably attached to the side face of the apparatus body; and
an engagement part with which the apparatus body is provided, the engagement part being detachably engageable with another end of the hook body, and
the hook body is engaged with the engagement part, and the hook body forms, together with the side face, a region through which the cable is inserted.

7. The medical measurement apparatus according to claim 1,
wherein the hook includes:
a hook body being U-shaped, the hook body forming, together with the side face of the apparatus body, an insertion region through which the cable is inserted; and
a flap openably and closably provided at part of the hook body,
from a state where the flap is open, the flap moves from a side of location of the insertion region to enclose the insertion region, and
from a state where the flap is closed, the flap moves from outside to release the insertion region.
